(19) United States
(12) Patent Application Publication
(10) Pub. No.: US 2005/0064007 A1
(43) Pub. Date: Mar. 24, 2005

(54) Surface-modified implants

(76) Inventors: Samuel G. Steinemann, Basel (CH); James Percival Simpson, Arisdorf (CH)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/501,957

(22) PCT Filed: Jan. 14, 2003
(86) PCT No.: PCT/CH03/00013

(30) Foreign Application Priority Data
Jan. 21, 2002 (CH) 88/02

(51) Int. Cl. A61F 2/00

(52) U.S. Cl. 424/423

(57) ABSTRACT

An osteogenic implant with improved osteointegration properties. In one embodiment said implant consists of titanium or a titanium base alloy and has an at least partially roughed-up surface. Said surface, in the hydroxylated state, is at least partially coated with a polypeptide, namely a transforming growth factor (TGF) or a systemic hormone, or with a mixture of such compounds.

The present invention relates to surface-modified osteogenic implants which are used for insertion into bones and which display considerably improved osteointegration properties, and to processes for the production thereof.

Implants which are used for insertion into bones, such as, for example, hip or knee joint prostheses or pins to be screwed into the jaw to construct artificial teeth, are known per se. Such implants preferably consist of titanium or titanium-based alloys such as, for example, titanium/zircon alloys, it being possible for the latter additionally to contain niobium, tantalum or other tissue-compatible metallic additions. The central properties of such implants are the strength of the anchoring in the bone and the period of time in which integration is achieved. Osteointegration accordingly means the frictionally solid and permanent connection between implant surface and bone tissue.

The firmness of the anchoring of the implant in the bone can be established by mechanical measurements, namely by measuring the force, whether as pulling, pushing, shearing or torque, which are necessary in order to extract or unscrew the implant anchored in the bone from its anchoring, i.e. bring about a break of the adhesion between the surface of the implant and the bone substance connected thereto. Such measurement methods are known per se and described, for example, in Brunski, Clinical Materials, Vol. 10, 1992, pp. 153-201. Measurements have shown only little anchoring of titanium implants with a smooth surface structure in the bone, whereas implants with a roughened surface afford a noticeably improved bone-implant connection in relation to their tenacity.

EP 0 388 576 therefore proposes applying to the implant surface, in a first step, a macro-roughness by means of sandblasting, and subsequently superimposing a micro-roughness on the latter by means of treatment in an acid bath. The implant surface can thus be roughened by means of sandblasting and subsequently treated with an etching agent, e.g. hydrofluoric acid or hydrochloric acid/sulfuric acid mixture. The surface provided with a defined roughness in this way is then cleaned with solvents and water and subjected to a sterilizing treatment.

The chemical state of the surface of titanium and titanium-based alloys is complex. It is assumed that the surface of titanium metal spontaneously oxidizes in air and water and that a reaction with water then takes place on the surface, that is to say in the outermost atomic layer of the oxide, with formation of hydroxyl groups. This surface containing hydroxyl groups is referred to in the literature as “hydroxylated” surface. See H. P. Boehm, Acidic and Basic Properties of Hydroxylated Metal Oxide Surfaces, Discussions Faraday Society, Vol. 52, 1971, pp. 264-275.

It has now been found that a hydroxylated surface of surface-oxidized titanium metal or oxidized titanium-based alloy has bioactive properties, because the metallic foreign body forms a frictional connection with the bone tissue, that is to say undergoes osteointegration. It has emerged, surprisingly, that such a hydroxylated and bioactive surface retains its activity over a longer period and unites with the bone substance to give a strong connection considerably more quickly than an identical surface which has not been treated according to the invention and is normally dried in the air, when this hydroxylated surface has been treated with a polypeptide, which represents (i) a transforming growth factor (TGF), for example a transforming growth factor beta (TGF-β) or an osteogenic growth peptide (OGP), or (ii) a systemic hormone, or with a mixture of such compounds, or this hydroxylated surface has been at least partially covered with such a compound or a mixture of such compounds. In this way, an osteogenic implant with improved osteointegration properties, in particular also with an accelerated anchoring reaction, is obtained, and the bioactivity of the hydroxylated implant surfaces treated according to the invention remains substantially unchanged until the implant is inserted.

The present invention is defined in the patent claims. The invention relates to an osteogenic implant made of a biocompatible material, the surface being at least partially covered with a polypeptide selected from the group of transforming growth factors (TGF) and systemic hormones, or a mixture of such compounds. The present invention relates in particular to a surface-modified osteogenic implant with improved osteointegration properties or with improved osteointegration, this implant consisting of titanium metal, a titanium-based alloy, a ceramic material, in particular an oxide ceramic, and preferably having an at least partially roughened surface, which surface has been treated with a polypeptide which represents a transforming growth factor (TGF) or a systemic hormone, or with a mixture of such compounds, and this hydroxylated surface has been at least partially covered with such a compound or a mixture of such compounds.

Transforming growth factor (TGF) is to be understood in particular as the group (subgroup) of the (i) transforming growth factors beta (TGF-β) and the group (subgroup) of the (ii) bone morphogenic proteins (BMP). Bone morphogenic proteins (BMP) are, for example, osteonectin, bone sialoprotein (BSP), osteopontin, osteocalcin, osteostatin, osteogenin, and osteo growth peptides (OGP).

This surface is preferably stored enclosed in a gas-tight and liquid-tight envelope and in an atmosphere which is inert for the implant surface, that is to say that no compounds which are able to impair the bioactivity of the implant surface are present inside the envelope.

The inside of the envelope is preferably filled with gases which are inert for the implant surface, such as, for example, oxygen, nitrogen, noble gases or a mixture of such gases. The inside of the envelope may, however, also be at least partially filled with pure water which optionally contains additives, in which case the amount of water present is at least such that wetting of the roughened implant surface is ensured. The remaining volume inside the envelope can be filled with gases which are inert for the implant surface, such as, for example, oxygen, nitrogen, noble gases or a mixture of such gases.

The pure water present inside the envelope preferably comprises as additive or additives at least one polypeptide, which represents a transforming growth factor (TGF) or a systemic hormone, or a mixture of such compounds, that is to say at least one compound which is used according to the invention for the treatment and at least partial covering of the implant surface.

The present invention also relates to processes for producing the implants according to the invention, and to the implants produced according to the invention.

The implants according to the invention preferably consist of a titanium-based alloy, preferably of a titanium/zircon alloy, it being possible for the latter additionally to contain niobium, tantalum or other tissue-compatible metallic additions. Ceramic materials, in particular oxide ceramics, can also be used, particularly preferably zirconium oxide-based ceramics. Implants of this type, their characteristics and the metallic materials used to produce them are known per se and described, for example, in J. Black, G. Hastings, Handbook of Biomaterials Properties, pages 135-200, published by Chapman & Hall, London, 1998. Ceramic materials are described for example in U.S. Pat. No. 6,165,925. The invention can be particularly advantageously applied for dental implants, that is to say pins which are to be screwed into the jaw for constructing artificial teeth.

The present invention also relates to a process for introducing an osteogenic implant of at least partially cylindrical shape into a cavity of a jaw bone, the bone surface, in the area of the cavity, being brought at least partially into contact with a polypeptide selected from the group of transforming growth factors (TGF) and systemic hormones, or a mixture of such compounds. This can be done, for example, by using a hydrogel containing a polypeptide selected from the group of transforming growth factors (TGF) and systemic hormones, or a mixture of such compounds. Such a hydrogel can be applied, for example, to the implant and/or into the cavity of the jaw bone, in particular in addition to the stated surface treatment of the implant. Further improved osteointegration can be achieved in this way.

Investigations have shown that adequate anchoring of an implant in the bone depends to a large extent on the surface characteristics of the implant, especially on the roughness. According to the present invention, the bioactivity of the surface treated according to the invention supplements synergistically the essentially physical effect of the surface roughness, resulting in a considerable improvement in osteointegration. The tooth implant according to the invention preferably has a macro-roughness such as, for example, a screw thread or recesses in the surface, which can be obtained for example by mechanical treatment and structuring, shotpeening or sandblasting. In addition, this roughened surface preferably has a superimposed micro-roughness, this micro-roughness preferably being produced by chemical etching of the surface or by means of electrochemical (electrolytic) treatment or by a combination of these processes. This results in a surface which is hydroxylated and at the same time also hydrophilic. This hydroxylated surface is treated according to the invention with a polypeptide which represents a transforming growth factor (TGF) or a systemic hormone, or with a mixture of such compounds, and this hydroxylated surface is at least partially covered with such a compound or a mixture of such compounds.

The hydroxylated surface can be produced for example by providing the surface with the desired roughness or texture, in particular by the implant surface being initially shotpeened, sandblasted and/or roughened by use of plasma technology, and subsequently treating the mechanically roughened surface with an electrolytic or chemical process until a hydroxylated and hydrophilic surface is produced. The implant is preferably etched with an inorganic acid or a mixture of inorganic acids, preferably with hydrofluoric acid, hydrochloric acid, sulfuric acid, nitric acid or a mixture of such acids, or else the surface is activated with hydrochloric acid, hydrogen peroxide and water in the ratio of about 1:1:5 by weight.

The procedure is preferably as follows:
the implant is shotpeened and subsequently etched with dilute hydrofluoric acid at room temperature and washed with pure distilled and CO₂-free water; or
the implant is sandblasted, e.g. with alumina particles having an average particle size of 0.1-0.25 mm or 0.25-0.5 mm and subsequently treated with a hydrochloric acid/sulfuric acid mixture at elevated temperature and washed with pure distilled and CO₂-free water; or
the implant is sandblasted with coarse particles, e.g. with a particle mixture as previously defined, and subsequently treated with a hydrochloric acid/nitric acid mixture and washed with pure distilled and CO₂-free water; or
the implant is treated with a mixture of hydrogen chloride, hydrogen peroxide and water in the ratio of about 1:1:5 by weight and washed with pure distilled and CO₂-free water; or
the implant is roughened by using plasma technology and subsequently hydroxylated in a mixture of hydrogen chloride, hydrogen peroxide and water in the ratio of about 1:1:5 by weight and washed with pure distilled and CO₂-free water; or
the implant is treated in an electrolytic process, the surface having previously been roughened mechanically where appropriate, and subsequently washed with pure distilled and CO₂-free water.

In all cases, the implant or its hydroxylated surface is treated according to the invention directly with a polypeptide which represents a transforming growth factor (TGF) or a systemic hormone, or with a mixture of such compounds. In particular, the implant or its hydroxylated surface is not treated with alcohol, acetone or another organic solvent or a disinfectant or exposed to the atmosphere or gaseous substances such as, for example, hydrocarbons, which are not inert for the hydroxylated and hydrophilic surface and would reduce or destroy for example the hydrophilic surface property. The “pure” water used in the process contains neither carbon dioxide nor vapors of hydrocarbons, and no alcohols such as methanol or ethanol, and no acetone or related ketones. However, it may comprise specific additives as described hereinafter.

The “pure” water used for washing is preferably water which has been distilled several times or prepared by inverse osmosis and which has preferably been prepared in an inert atmosphere, that is to say, for example, under reduced pressure, in a nitrogen or noble gas atmosphere. In particular, the pure water has an electrical resistance of at least 2 mohmcm (electrical resistance >2 mohmcm) and a total organic carbon content (total organic carbon, TOC) not exceeding 10 ppb (<10 ppb).

Subsequent to the washing process, the resulting implant is preferably stored in pure water which may optionally comprise additives. The resulting implant is preferably stored in a closed envelope which is filled with a gas which is inert for the implant surface, for example nitrogen, oxygen or noble gas, such as, for example, argon, and/or in pure water which optionally contains additives, until further processing according to the invention. The envelope is preferably virtually impermeable for gases and liquids.

The implant which has a hydroxylated surface, or the hydroxylated surface of the implant, is treated according to the invention in the hydroxylated state with a polypeptide which represents a transforming growth factor (TGF) or a systemic hormone, or with a mixture of such compounds, and is at least partially covered with such a compound or a mixture of such compounds.

As has already been mentioned, transforming growth factor (TGF) is to be understood in particular as the group of the (i) transforming growth factors beta (TGF-β) and the group of the (ii) bone morphogenic proteins (BMP). Bone morphogenic proteins (BMP) are, for example, osteonectin, bone sialoprotein (BSP), osteopontin, osteocalcin, osteostatin, osteogenin, and osteo growth peptides (OGP).

Examples of proteins and polypeptides from the group of transforming growth factor beta (TGF-β) are for example the factors TGF-β1, TGF-β2, TGF-β3, TGF-β4 and TGF-β5, which are described, for example, in A. B. Roberts, M. B. Sporn, Handbook of Experimental Pharmacology, 95 (1990), pages 419-472, or in D. M. Kingsley, Genes and Development 8 (1994), pages 133-146, and the works cited there. These compounds are incorporated herein by reference.

Examples from the group of bone morphogenic proteins (BMP) are the proteins BMP-2 (BMP-2a), BMP-3, BMP-4 (BMP-2b), BMP-5, BMP-6, BMP-7 (OP-1), BMP-8 (OP-2), BMP-9, BMP-10, BMP-11, BMP-12, BMP-13 which are described, for example, in J. M. Wozney et al., Science 242 (1988), pages 1528-1534; A. J. Celeste et al., Proc. Natl. Acad. Sci. USA 87 (1990), pages 9843-9847; E. Özkaynak et al., J. Biol. Chem. 267 (1992), pages 25220-25227; Takao et al., Biochem. Biophys. Res. Com. 219 (1996), pages 656-662; WO 93/00432; WO 94/26893; WO 94/26892; WO 95/16035, and the works cited therein. These compounds are incorporated herein by reference.

Examples of osteocalcins are:
Osteocalcin (7-19) (human): H-Gly-Ala-Pro-Val-Pro-Tyr-Pro-Asp-Pro-Leu-Glu-Pro-Arg-OH;
Osteocalcin (37-49) (human): H-Gly-Phe-Gln-Glu-Ala-Tyr-Arg-Arg-Phe-Tyr-Gly-Pro-Val-OH;
(Tyr³⁸, Phe^{42,46}) Osteocalcin (38-49): H-Tyr-Gln-Glu-Ala-Phe-Arg-Arg-Phe-Gly-Pro-Val-OH;
Osteocalcin (1-49) (human): H-Tyr-Leu-Tyr-Gln-Trp-Leu-Gly-Ala-Pro-Val-Pro-Tyr-Pro-Asp-Pro-Leu-Gla-Pro-Arg-Arg-Gla-Val-Cys -Gla-Leu-Asn-Pro-Asp-Cys-Asp-Glu-Leu-Ala-Asp-His-Ile-Gly-Phe-Gln-Gln-Ala -Tyr-Arg-Arg-Phe-Tyr-Gly-Pro-Val-OH (Gla gamma-carboxy-L-glutamyl).
Osteogenic growth peptides (OGP) are known. Such a peptide with 14 amino acids corresponds, for example, to the formula: H-Ala-Leu-Lys-Arg-Gln-Gly-Arg-Thr-Leu-Tyr-Gly-Phe-Gly-Gly-OH.

Systemic hormones are known per se and can be used in the form known per se. Systemic hormones are, for example, the compounds designated as 1,25-(OH)₂D₃ or as 1α,1,25(OH)₂D₃ or as 24,25-(OH)₂D₃. Such systemic hormones are described, for example, in Boyan B. D. et al., Journal of Biological Chemistry, 264, pages 11879-11888 (1989). The systemic hormones mentioned there are incorporated herein by reference.

Of the polypeptides which represent a transforming growth factor (TGF) or a systemic hormone, preference is given to those which contain at least one residue of an amino acid with a heterocyclic ring, for example the residue of proline (Pro), hydroxyproline (Hypro), tryptophan (Try) or histidine (His).

Methods for characterizing and analyzing metal surfaces are known per se. These methods can also be used for measuring and checking or monitoring the covering density. Such analysis methods known per se are, for example, infrared spectroscopy, laser desorption mass spectroscopy (LDMS), X-ray-excited photoelectron spectroscopy (XPS), matrix-assisted laser desorption ion mass spectroscopy (MALDI), time of flight secondary ion mass spectroscopy (TOFSIMS), electron and ion microanalysis, optical waveguide light mode spectroscopy (OWLS) or X-ray photoelectron diffraction (XPD). It can be used to measure for example the titanium atoms or hydroxyl groups available on the metal surface. The metal atoms or hydroxyl groups available on the metal surface ordinarily provide the maximum covering density of the surface with a monomolecular layer (“monolayer”) . The stated analysis methods known per se can be used to measure the concentration and the thickness of the monomolecular layer, which depends in particular on the chemical composition of the metal surface, the pretreatment thereof and the chemisorbed compound. Thus, for example, titanium oxide has about four to five reactive groups, with an acidic or basic reaction, per nm² of surface. This means that the surface of titanium oxide can be covered with about four molecules of an amino acid or polyamino acid per nm² of surface. It is preferred according to the invention for there to be only about 5%-70% coverage, based on the maximum coverage of the metal surface with a monomolecular layer of the stated compound. It is particularly preferred according to the invention for the coverage to be about 8%-50%, and in particular about 8%-20%, based on the maximum coverage of the metal surface with monomolecular layer. In this sense, the metal surface continues to remain at least partially hydroxylated, through the remaining “free” hydroxyl groups, so that a combination of the two effects affords an implant with very good osteointegration properties.

The polypeptide, which represents an osteogenic growth peptide (OGP) or a transforming growth factor (TGF) or an osteocalcin, or the mixture of these compounds, is applied to the hydroxylated surface of the implant in a suitable method, for example from aqueous solution or from an organic solvent or else by means of spraying with the pure compound or the pure compound mixture. The compound is thus adsorbed and bound by the hydroxylated surface. Bound means in this connection that it cannot be removed directly by rinsing with water. It is sufficient in this connection for the compound to be brought into contact with the hydroxylated metal surface in aqueous or organic solution of very low concentration, depending on the compound, in a concentration of the order of 0.01 μmol/l (micromole per liter) or higher, for example 0.01 μmol/l to about 100 μmol/l, preferably 0.1 μmol/l to about 10 μmol/l, preferably about 1 μmol/l, in order to produce the desired coverage. These concentration limits are, however, not critical. The covering density of the surface which is achieved with said compounds is determined in particular by the concentration thereof in the liquid carrier, the contact time and the contact temperature, and the acid values (pH values) used.

In this sense, the present invention also relates to a process for producing an implant according to the invention, by the implant surface being shotpeened, sandblasted and/or roughened by use of plasma technology, wherein subsequently
(i) the surface which has been roughened mechanically or by plasma technology is treated with an electrolytic or chemical etching process until a hydroxylated surface has been produced, preferably with an inorganic acid or a mixture of inorganic acids, preferably with hydrofluoric acid, hydrochloric acid, sulfuric acid, nitric acid, or a mixture of such acids, or hydrogen chloride, hydrogen peroxide and water in the ratio of about 1:1:5 by weight; and
(ii) the surface is treated and at least partially covered with a polypeptide which represents an osteogenic growth peptide (OGP) or a transforming growth factor (TGF) or an osteocalcin, or with a mixture of such compounds.

The coverage of the hydroxylated metal surface with said compound, or with said compound mixture, can be explained by a chemisorption or by a chemical binding. This means that a reactive group of the added compound enters into a condensation reaction with the hydroxyl group present on the metal surface, for example in accordance with the formula: ≡TiOH+—CH₂C(O)OH→≡TiOC(O)CH₂—+H₂O, where ≡Ti— is a metal ion on the metal surface. An amphoteric character may be attributed to the surface depending on the acid value of the electrolyte surrounding the surface, there being an interaction between the acid in the electrolyte and the hydroxyl with a basic reaction on the oxide surface, or the anion in the electrolyte and the hydroxyl with an acidic reaction in the oxide. The surface reactions can be explained through the formation of covalent bonds, electrostatic effects and/or the formation of hydrogen bridges. The present invention is not, however, tied to these explanations. The decisive fact is that the surface treatment described here preserves and improves the bioactivity of the hydroxylated surface.

In order to bind the polypeptide to the metal surface, the procedure is preferably such that the compound is applied from aqueous or organic solution, preferably from aqueous solution, by wetting, or by spraying with the pure compound, to the surface. There is, where appropriate, heating to a temperature of about 40° C. to 70° C., where appropriate under pressure. The binding of the compound to the surface can likewise be promoted with UV radiation. A further method consists in applying the compound, depending on the nature of the compound, from aqueous acidic or basic solution to the surface. In this case, the solution preferably has an acid value (pH value) of between 2 and 4 or between 8 and 11. The implant can subsequently be treated where appropriate with UV radiation.

The implant according to the invention, or at least its covered surface according to the invention, is preferably enclosed in a gas-tight and liquid-tight envelope, there being no compounds inside the envelope which are able to impair the bioactivity of the implant surface, that is to say are inert for the implant surface. This gas-tight and liquid-tight envelope is preferably a sealed ampule made of glass, metal, a synthetic polymer or another gas-tight and liquid-tight material, or a combination of these materials. The metal is preferably in the form of a thin metal sheet, it being possible to combine polymeric materials and metallic sheets, but also glass, in a manner known per se with one another to give a suitable packaging.

It is preferred for there to be an inert atmosphere inside the envelope and for it to be filled with an inert gas and/or at least partially with pure water, which optionally contains additives. A suitable additive which can be added according to the invention to the pure water for improved storage of the implant is, in particular, a polypeptide which represents an osteogenic growth peptide (OGP) or a transforming growth factor (TGF) or an osteocalcin, or a mixture of such compounds, and in particular the same compound or the same mixture of compounds with which the implant surface has been covered. In this case, the pure water contains said compound or the mixture of compounds preferably in a concentration in the range from about 0.01 μmol/l to 100 μmol/l (micromole per liter), preferably about 0.1 μmol/l to 10 μmol/l, and preferably in a concentration of about 1 μmol/l.

Further suitable additions which can be added according to the invention to the pure water are monovalent alkali metal cations such as Na⁺ or K⁺, or a mixture of Na⁺ and K⁺, with appropriate anions in the form of inorganic salts, such as, for example, sodium chloride, potassium chloride, sodium or potassium chlorate, sodium or potassium nitrate, sodium or potassium phosphate or a mixture of such salts. It is likewise also possible to add divalent cations in the form of water-soluble inorganic salts. Suitable cations are, in particular, Mg⁺², Ca⁺², Sr⁺² and/or Mn⁺² in the form of the chlorides, chlorates, nitrates or mixtures thereof. Suitable inorganic anions are also phosphate and phosphonate anions, by which are meant in each case also monoorthophosphate anions and diorthophosphate anions, and monoorthophosphonate anions and diorthophosphonate anions, in combination with the cations mentioned. In clinical application, such implants enclosed in an ampule can be used directly without any further treatment.

Preferred inorganic cations and anions are those which already occur in body fluid, especially in the respective physiological concentration and with a physiological acid value in the range of preferably 4 to 9 and preferably with an acid value in the range of 6 to 8. Preferred cations are Na⁺, K⁺, Mg⁺² and Ca⁺². The preferred anion is Cl⁻. The total amount of said cations and anions is preferably in each case in the range from about 50 mEq/l to 250 mEq/l, preferably about 100 mEq/l to 200 mEq/l, and preferably about 150 mEq/l. Here, Eq/l means (formula) equivalent weight, and Eq/l corresponds to the atomic weight of the formula unit divided by the valency. mEq/l means milliequivalent weight per liter. If the envelope contains divalent cations, in particular Mg⁺², Ca⁺², Sr⁺² and/or Mn⁺², alone or in combination with the monovalent cations mentioned, then the total amount of the divalent cations present is preferably in the range from 1 mEq/l to 20 mEq/l. It is likewise possible for the abovementioned organic compounds to be present in a mixture with the stated inorganic salts dissolved in pure water, in which case the stated concentrations for the additions which are present still apply and are usually sufficient.

Methods for measuring the effective surface area of a metallic body are known per se. Thus, for example, electrochemical measurement methods are known and are described in detail in P. W. Atkins, Physical Chemistry, Oxford University Press, 1994. It is also possible to obtain the effective surface area from roughness measurements as the square of the hybrid parameter Lᵣ, i.e. the square of the profile-length ratio. The parameter Lᵣ is defined in the standard DIN 4762 as the ratio of the length of the extended two-dimensional profile and of the measured distance. However, the precondition for the latter measurement is that the vertical and lateral resolution of the measurement method is less than 1 μm and is in fact close to 0.1 μm.

The reference area for all these measurement methods is the flat polished metal surface. The measured values for the roughened surface compared with those on the flat and polished surface indicate how much greater the roughened surface is, compared with the flat and polished surface. In vitro investigations with bone cells and in vivo histomorphometric investigations on implants according to the invention indicate that the osteogenic properties of the implants according to the invention are particularly high when the roughened surface is preferably at least 1.5 times and preferably at least twice as large as the comparable flat and polished surface. The roughened implant surface is preferably at least 2 to 12 times as large, and preferably about 2.5 to 6 times as large, as the comparable flat and polished surface.

Industrially produced surfaces of titanium and titanium alloys for processing in laboratories and clinics usually have impurities which consist essentially of carbon compounds and traces of nitrogen, calcium, sulfur, phosphorus and silicon. These impurities are concentrated in the outermost metal oxide layer. The hydroxylated and hydrophilic implant surface preferably contains not more than 20 atom % carbon measured by spectroscopic methods such as XPS or AES or other spectroscopic methods known per se.

The following examples illustrate the invention.

### EXAMPLE 1

A) A conventional form of a tooth implant in the form of a screw with a diameter of 4 mm and a length of 10 mm was produced. The basic form was obtained by removing material by turning and milling the cylindrical preform in a manner known per se. The surface to be inserted into the bone was then provided with a macro-roughness as described in EP 0 388 576 by sandblasting it with particles having an average particle size of 0.25-0.50 mm. Subsequently, the roughened surface (macro-roughness) was treated with an aqueous hydrochloric acid/sulfuric acid mixture with an HCl:H₂SO₄:H₂O ratio of 2:1:1 at a temperature above 80° C. for about five minutes, to result in a ratio of the roughened implant surface to the comparable polished surface of 3.6, measured by voltametry in aqueous electrolyte with 0.15M NaCl (corresponding to a ratio of 3.9 measured by impedance spectrometry in 0.1 molar Na₂SO₄ electrolyte). The implant formed in this way was washed with pure water.

B) Subsequently, the implant obtained in section A) was left in a solution consisting of pure water, which contained the osteogenic growth peptide (OGP) of the formula: H-Ala-Leu-Lys-Arg-Gln-Gly-Arg-Thr-Leu-Tyr-Phe-Gly-Gly-OH in a concentration of 100 μmol per liter, under nitrogen for 24 hours. The implant was removed and washed with pure water under nitrogen. Measurement revealed a coverage of about 10% of the metal surface. The implant was subsequently

a) sealed directly in a glass ampule which was filled with pure water, opened after 4 weeks, and implanted;

b) sealed directly in a glass ampule which was filled with pure water which was adjusted to pH=9 with 0.2M sodium bicarbonate and contained the pentapeptide Gly-Arg-Gly-Asp-Ser in a concentration of 1 μmol/l. The glass ampule was opened after 4 weeks, briefly washed in isotonic saline, and implanted;

c) after completion of the treatment as in section A), dried in atmospheric air and implanted (comparative test).

The implants obtained as in tests a), b) and c) were implanted into the upper jaw of a minipig. The anchoring was measured as the loosening torque in Ncm (mean values) after 2 weeks, after 3 weeks, and after 4 weeks. The results in tests a) and b) (implants according to the invention), and the corresponding loosening torques for the stated incorporation times, are distinctly higher than those of test c), which shows shorter incorporation times and an accelerated osteointegration.

### EXAMPLE 2

Example 1 was repeated, but with the proviso that the osteogenic growth peptide (OGP) used in section B) was replaced by osteocalcin (7-19) (human) of the formula: H-Gly-Ala-Pro-Val-Pro-Tyr-Pro-Asp-Pro-Leu-Glu-Pro-Arg-OH. Results analogous to those according to example 1, section B were obtained.

### EXAMPLE 3

Example 1 was repeated, with the proviso that the acid treatment according to example 1, section A is followed by introduction into pure water containing 0.15 mol/l NaCl and where appropriate 0.005 mol/l CaCl₂. The osteogenic growth peptide (OGP) or osteocalcin (7-19) (human) of the formula: H-Gly-Ala-Pro-Val-Pro-Tyr-Pro-Asp-Pro-Leu-Glu-Pro-Arg-OH in a concentration of 100 micromol/l was added to this electrolyte. The whole was sealed in a glass ampule under nitrogen. The glass ampule was opened after 4 weeks, the implant obtained was removed and was implanted into the upper jaw of a minipig without any further treatment, i.e. without drying or washing. The anchoring was measured as the loosening torque in Ncm (mean values) after 2 weeks, after 3 weeks, and after 4 weeks. Results analogous to those according to example 1 were obtained.

1-27. Cancel

28. An osteogenic implant comprising an implant made of titanium metal, having a surface covered with a polypeptide at a rate of 5 to 70%, preferably 8% to 20%, based on a maximum coverage of the metal surface with a monomolecular layer, wherein the polypeptide is selected from the group consisting of one or more of transforming growth factors (TGF) and systemic hormones.

29. The implant of claim 28, having an at least partially roughened surface, which surface is at least partially covered, in the hydroxylated state, with a polypeptide selected from the group consisting of one or more of transforming growth factors (TGF) and systemic hormones.

30. The implant of claim 29, having a macro-roughness, and a micro-roughness superposed on the macro-roughness, said micro-roughness being produced by chemical etching of the surface and/or by means of electrolytic treatment, preferably by etching with an inorganic acid or a mixture of inorganic acids, preferably with hydrofluoric acid, hydrochloric acid, sulfuric acid, nitric acid or a mixture of such acids, or else by treating the surface with hydrochloric acid, hydrogen peroxide and water in a ratio of about 1:1:5 by weight.

31. The implant of claim 28, wherein the transforming growth factor (TGF) is selected from the group consisting of one or more of (i) transforming growth factors beta (TGF-β) and (ii) bone morphogenic proteins (BMP).

32. The implant of claim 31, wherein the transforming growth factor beta (TGF-β) is selected from the group consisting of one or more of TGF-β1, TGF-β2, TGF-β3, TGF-β4 and TGF-β5.

33. The implant of claim 31, wherein the TGF is a bone morphogenic protein (BMP) selected from the group consisting of one or more of BMP-2 (BMP-2a), BMP-3, BMP-4(BMP-2b), BMP-5, BMP-6, BMP-7 (OP-1), BMP-8 (OP-2), BMP-9, BMP-10, BMP-11, BMP-12, and BMP-13.

34. The implant of claim 31, wherein the TGF is a bone morphogenic protein (BMP) selected from the group consisting of one or more of osteonectin, bone sialoprotein (BSP), osteopontin, osteocalcin, osteostatin, osteogenin, and osteo growth peptides (OGP).

35. The implant of claim 34, wherein the osteocalcin has a formula: H-Gly-Ala-Pro-Val-Pro-Tyr-Pro-Asp-Pro-Leu-Glu-Pro-Arg-OH.

36. The implant of claim 34, wherein the osteocalcin has a formula: H-Gly-Phe-Gln-Glu-Ala-Tyr-Arg-Arg-Phe-Tyr-Gly-Pro-Val-OH.

37. The implant of claim 34, wherein the osteocalcin has a formula: H-Tyr-Gln-Glu-Ala-Phe-Arg-Arg-Phe-Gly-Pro-Val-OH.

38. The implant of claim 34, wherein the osteocalcin has a formula: H-Tyr-Leu-Tyr-Gln-Trp-Leu-Gly-Ala-Pro-Val-Pro-Tyr-Pro-Asp-Pro -Leu-Gla-Pro-Arg-Arg-Gla -Val-Cys-Gla-Leu-Asn-Pro-Asp-Cys-Asp-Glu-Leu-Ala-Asp-His-Ile-Gly-Phe-Gln-Gln-Ala -Tyr-Arg-Arg-Phe-Tyr-Gly-Pro-Val-OH.

39. The implant of claim 34, wherein the osteogenic growth peptide (OGP) has a formula: H-Ala-Leu-Lys-Arg-Gln-Gly-Arg-Thr-Leu-Tyr-Gly-Phe-Gly-Gly-OH.

40. The implant of claim 28, wherein the polypeptide contains at least one residue of an amino acid with a heterocyclic ring, preferably the residue of proline (Pro), hydroxyproline (Hypro), tryptophan (Try) or histidine (His).

41. The implant of claim 28, wherein the systemic hormone comprises one or more of 1,25-(OH)₂D₃, 1α,1,25(OH)₂D₃ and 24,25-(OH)₂D₃.

42. The implant of claim 28, wherein the implant, or at least its covered surface, is enclosed in a gas-tight and liquid-tight envelope which is filled with a gas which is inert for the implant surface, preferably with nitrogen, oxygen or a noble gas and/or at least partially with pure water, which optionally contains additives.

43. The implant of claim 42, wherein the pure water in the envelope contains a polypeptide comprising one or more of a transforming growth factor (TGF) and a systemic hormone, preferably the same polypeptide with which the implant surface is covered.

44. The implant of claim 43, wherein the pure water contains the polypeptide in a concentration in the range from 0.01 μmol/l to 100 μmol/l, preferably 0.1 μmol/l to 10 μmol/l, and preferably in a concentration of about 1 μmol/l.

45. The implant of claim 44, wherein the pure water contains inorganic salts in the form of monovalent alkali metal cations, preferably Na⁺ or K⁺, or a mixture of Na⁺ and K⁺, with anions and/or divalent cations in the form of water-soluble inorganic salts, preferably Mg⁺², Ca⁺², Sr⁺² and/or Mn⁺² in the form of the chlorides, chlorates, nitrates, phosphates and/or phosphonates.

46. The implant of claim 42, wherein the pure water contains inorganic salts in a total amount of said cations and anions in each case in a range from 50 mEq/l to 250 mEq/l, preferably 100 mEq/l to 200 mEq/l, and preferably in an amount of about 150 mEq/l.

47. A process for producing an implant of claim 28, wherein the implant surface is mechanically roughened by being shotpeened or sandblasted and/or roughened by use of plasma technology, wherein subsequently
(i) the surface which has been roughened mechanically or by plasma technology is treated with an electrolytic or chemical etching process until a hydroxylated surface has been produced, preferably with an inorganic acid or a mixture of inorganic acids, preferably with hydrofluoric acid, hydrochloric acid, sulfuric acid, nitric acid, or a mixture of such acids, or hydrogen chloride, hydrogen peroxide and water in a ratio of about 1:1:5 by weight; and
(iii) the surface is at least partially covered with a polypeptide comprising one or more of an osteogenic growth peptide (OGP), a transforming growth factor (TGF) and an osteocalcin.

48. The process of claim 47, wherein the polypeptide is brought into contact with the hydroxylated metal surface in aqueous solution at a concentration of at least 10 μmol/l (micromole per liter).

49. The implant produced by the process of claim 47.

50. The implant of claim 28, wherein it is a dental implant.

51. A process for introducing an osteogenic dental implant of at least partially cylindrical shape into a cavity of a jaw bone, wherein the bone surface, in the area of the cavity, is brought at least partially into contact with a polypeptide selected from the group consisting of one or more of transforming growth factors (TGF) and systemic hormones, wherein the metal surface is covered with the polypeptide at a rate of 5 to 70%, preferably 8% to 20%, based on a maximum coverage of the metal surface with a monomolecular layer.
